(19) United States
(12) Patent Application Publication
(10) Pub. No.: US 2024/0366523 A1
(43) Pub. Date: Nov. 7, 2024

(54) PATCH AND PRODUCTION METHOD THEREOF

(71) Applicant: KANEKA CORPORATION, Osaka (JP)

(72) Inventors: Masaya MIZUTANI, Hyogo (JP); Hiroyuki OGINO, Hyogo (JP); Masaoki GOTO, Hyogo (JP)

(21) Appl. No.: 18/683,624

(22) PCT Filed: Aug. 10, 2022
(86) PCT No.: PCT/JP2022/030654

§ 371 Date: Feb. 14, 2024

(30) Foreign Application Priority Data
Aug. 17, 2021 (JP) 2021-133024
Aug. 17, 2021 (JP) 2021-133025

(51) Int. Cl. A61K 9/70; A61K 31/167; A61K 31/245; A61K 47/06

(52) U.S. Cl. CPC A61K 9/7053; A61K 31/167; A61K 31/245; A61K 47/06

(57) ABSTRACT

A patch includes an adhesive layer that includes (a) a drug, (b) a thermoplastic elastomer, and (c) non-volatile hydrocarbon oil having a kinematic viscosity of 60 mm²/s or less at 40° C. The (a) drug includes (i) an amino group that may have a substituent, and (ii) at least one functional group selected from the group consisting of an ester group, an amide group, an ether group, a thioether group, and an amino group different from the (i) amino group, all of which may have a substituent. The (a) has a structure, in which the (i) amino group, and the (ii) at least one functional group are bonded via a C1-C3 hydrocarbon chain. The number of hydroxyl groups per molecule of the (a) is 4 or less. An amount of the (c) is 230 parts by mass or less relative to 100 parts by mass of the (b).

### TECHNICAL FIELD

The present invention relates to a patch and a production method thereof.

### BACKGROUND ART

For transdermal delivery of a drug, the drug is blended in an adhesive base to form a patch.

In recent years, a tape having excellent adhesion has been more commonly used as a patch than a poultice that contains a large amount of water in a patch. A thermoplastic elastomer is also widely used as an adhesive base polymer for the tape.

In order to secure sufficient adhesion as a patch, non-volatile hydrocarbon oil, such as liquid paraffin, may be used as a plasticizer (softening agent) for the thermoplastic elastomer. For example, it is disclosed in PTL 1 that non-volatile hydrocarbon oil having a high viscosity is suitable for realizing adequate adhesion and drug permeation through skin.

### CITATION LIST

### Patent Literature

[PTL 1] Japanese Patent Application Laid-Open (JP-A) No. 2015-113339

### SUMMERY OF INVENTION

### Problem to be Solved by Invention

When the present inventors produced a patch using a drug and the above described non-volatile hydrocarbon oil having a high viscosity, it was found that a phenomenon where a support was peeled from an adhesive layer of the patch (delamination or interlayer detachment, may be referred to as delamination hereinafter) occurred.

Accordingly, the present invention aims at solving the above-described various problems existing in the related art and achieving the following object. An object of the present invention is to provide a patch that suppresses delamination.

### Means for Solving the Problems

The present inventors have diligently conducted research to achieve the above-mentioned object. As a result, the present inventors have the following insights. A patch suppressing delamination can be provided when the patch includes an adhesive layer that includes (a) a drug, (b) a thermoplastic elastomer, and (c) non-volatile hydrocarbon oil having a kinematic viscosity of 70 mm²/s or less at 40° C., where the (a) drug includes (i) an amino group that may have a substituent and (ii) at least one functional group selected from the group consisting of an ester group, an amide group, an ether group, a ketone group, a thioether group, and an amino group different from the (i) amino group, all of which may have a substituent; the (a) drug has a structure, in which the (i) amino group, and the (ii) at least one functional group are bonded via a C1-C3 hydrocarbon chain; the number of hydroxyl groups per molecule of the (a) drug is 4 or less; and an amount of the (c) non-volatile hydrocarbon oil is 230 parts by mass or less relative to 100 parts by mass of the (b) thermoplastic elastomer.

The present invention has been accomplished based on the above-described insights of the present inventors, and means for solving the above-described problems are as follows.
<1> A patch includes an adhesive layer. The adhesive layer includes (a) a drug, (b) a thermoplastic elastomer, and (c) non-volatile hydrocarbon oil having a kinematic viscosity of 70 mm²/s or less at 40° C. The (a) drug includes (i) an amino group that may have a substituent and (ii) at least one functional group selected from the group consisting of an ester group, an amide group, an ether group, a ketone group, a thioether group, and an amino group different from the (i) amino group, all of which may have a substituent. The (a) drug has a structure, in which the (i) amino group, and the (ii) at least one functional group are bonded via a C1-C3 hydrocarbon chain. The number of hydroxyl groups per molecule of the (a) drug is 4 or less. An amount of the (c) non-volatile hydrocarbon oil is 230 parts by mass or less relative to 100 parts by mass of the (b) thermoplastic elastomer.
<2> A production method of the patch of <1> includes mixing (a) a drug, (b) a thermoplastic elastomer, and (c) non-volatile hydrocarbon oil having a kinematic viscosity of 70 mm²/s or less at 40° C. The (a) drug includes (i) an amino group that may have a substituent and (ii) at least one functional group selected from the group consisting of an ester group, an amide group, an ether group, a ketone group, a thioether group, and an amino group different from the (i) amino group, all of which may have a substituent. The (a) drug has a structure, in which the (i) amino group, and the (ii) at least one functional group are bonded via a C1-C3 hydrocarbon chain. The number of hydroxyl groups per molecule of the (a) drug is 4 or less. An amount of the (c) non-volatile hydrocarbon oil is 230 parts by mass or less relative to 100 parts by mass of the (b) thermoplastic elastomer.
<3> An anti-delamination agent includes (a) a drug, (b) a thermoplastic elastomer, and (c) non-volatile hydrocarbon oil having a kinematic viscosity of 70 mm²/s or less at 40° C. The (a) drug includes (i) an amino group that may have a substituent and (ii) at least one functional group selected from the group consisting of an ester group, an amide group, an ether group, a ketone group, a thioether group, and an amino group different from the (i) amino group, all of which may have a substituent. The (a) drug has a structure, in which the (i) amino group, and the (ii) at least one functional group are bonded via a C1-C3 hydrocarbon chain. The number of hydroxyl groups per molecule of the (a) drug is 4 or less. An amount of the (c) non-volatile hydrocarbon oil is 230 parts by mass or less relative to 100 parts by mass of the (b) thermoplastic elastomer.
<4> A method for suppressing delamination of a patch includes using the anti-delamination agent of <3> for a patch.

### Effect of Invention

According to the present invention, the above-described various problems existing in the related art can be solved, the above-described object can be achieved, and a patch that suppresses delamination.

### MODES FOR CARRYING OUT THE INVENTION

### Patch

The patch includes an adhesive layer, and may further include other elements.

A structure of the patch is not particularly limited, and may be appropriately selected according to the intended purpose. Examples of the structure of the patch include a matrix patch, a reservoir patch, and the like. The structure of the patch is preferably a matrix patch in view of easiness of production of a patch [symbol] [symbol] .

### <Adhesive Layer>

The adhesive layer includes (a) a drug, (b) a thermoplastic elastomer, and (c) non-volatile hydrocarbon oil having a kinematic viscosity of 70 mm²/s or less at 40° C. The adhesive may further include other components.

The adhesive layer is a mixture of the above-mentioned constituent components, and a solvent used during production is preferably 0.5% by mass or less, more preferably 0% by mass, relative to 100% by mass of a total amount of the constituent components of the adhesive layer.

### (a) Drug

The (a) drug includes at least (i) an amino group that may have a substituent and (ii) at least one functional group selected from an ester group, an amide group, an ether group, a ketone group, a thioether group, and an amino group different from the (i) amino group, all of which may have a substituent. The chemical structure of the (a) drug may further include other functional groups.

In view of suppression of delamination, among the above-listed examples, the (a) drug preferably includes (i) an amino group that may have a substituent and (ii) at least one selected from at least one functional group selected from an ester group, an amide group, and an amino group different from the (i) amino group, all of which may have a substituent, more preferably includes (i) an amino group that may have a substituent and (ii) an ester group or amide group that may have a substituent, and yet more preferably includes (i) an amino group that may have a substituent and (ii) an ester group that may have a substituent.

The (a) drug has a structure, in which the (i) amino group and the (ii) at least one functional group are bonded via a C1-C3 hydrocarbon chain, where the number of hydroxyl groups per molecule of the (a) drug is 4 or less.

Among the above-listed examples, the (a) drug preferably has a structure, in which the (i) amino group and the (ii) at least one functional group are bonded via a C1 or C2 hydrocarbon chain, in view of suppression of delamination.

The amino group that may have a substituent is not particularly limited, and may be appropriately selected according to the intended purpose. Examples of the amino group that may have a substituent include —NH₂ groups, methylamino groups, ethylamino groups, propylamino groups, butylamino groups, pentylamino groups, hexylamino groups, isopropylamino groups, tert-butylamino groups, 1-methylethylamino groups, diethylamino groups, dimethylamino groups, ethylphenylamino groups, 4-diethylaminophenyl groups, triphenylamine groups, diphenylamino groups, piperidine groups, quinoline groups, isoquinoline groups, indole groups, carbazole groups, pyridine groups, pyrrole groups, aziridine groups, azetidine groups, pyrrolidine groups, and the like. The above-listed groups may further have a substituent.

The substituent is not particularly limited, and may be appropriately selected according to the intended purpose. Examples of the substituent include halogen atoms, C1-C10 alkyl groups, and the like. Two sites in each of the C1-C10 alkyl groups may be bonded to each other to form a cyclic structure.

Among the above-listed examples, a propylamino group, a diethylamino group, a dimethylamino group, a tert-butylamino group, a piperazine group, a piperidine group, a butylpiperidine group, or a quinoline group is preferred.

An amine from which the amino group is derived may be an aliphatic amine, an aromatic amine, a heterocyclic amine, an imine, or an enamine. Moreover, an amine from which the amino group is derived may be a primary amine, a secondary amine, a tertiary amine, a linear amine, or a cyclic amine.

The ester group that may have a substituent is not particularly limited, and may be appropriately selected according to the intended purpose. Examples of the ester group that may have a substituent include —COO— groups, methyl ester groups, ethyl ester groups, propyl ester groups, butyl ester groups, pentyl ester groups, hexyl ester groups, isopropyl ester groups, phenyl ester groups, and the like. The above-listed examples may further include a substituent.

The substituent is not particularly limited, and may be appropriately selected according to the intended purpose. Examples of the substituent include halogen atoms, C1-C10 alkyl groups, and the like. Two sites in each of the C1-C10 alkyl groups may be bonded to each other to form a cyclic structure.

Among the above-listed examples, a —COO— group or a phenyl ester group is preferred.

The amide group that may have a substituent is not particularly limited, and may be appropriately selected according to the intended purpose. Examples of the amide group that may have a substituent include groups —CONH— groups, methyl amide groups, ethyl amide groups, propyl amide groups, butyl amide groups, pentyl amide groups, hexyl amide groups, isopropyl amide groups, phenyl amide groups, and the like. The above-listed examples may further include a substituent.

The substituent is not particularly limited, and may be appropriately selected according to the intended purpose. Examples of the substituent include halogen atoms, C1-C10 alkyl groups, and the like. Two sites in each of the C1-C10 alkyl groups may be bonded to each other to form a cyclic structure.

Among the above-listed examples, a —CONH— group or a phenyl amide group is preferred.

The ether group that may have a substituent is not particularly limited, and may be appropriately selected according to the intended purpose. Examples of the ether group that may have a substituent include —O— groups, ethyl ether groups, butyl ether groups, aryl ether groups, epoxy groups, oxetanyl groups, tetrahydrofuranyl groups, tetrahydropyranyl groups, and the like. The above-listed examples may further include a substituent.

The substituent is not particularly limited, and may be appropriately selected according to the intended purpose. Examples of the substituent include halogen atoms, C1-C10 alkyl groups, thiophenyl groups, and the like. Two sites in each of the C1-C10 alkyl groups may be bonded to each other to form a cyclic structure. Each of the thiophenyl groups may form a condensed ring with a cyclic ether group.

Among the above-listed examples, a tetrahydropyranyl group is preferred.

The ketone group that may have a substituent is not particularly limited, and may be appropriately selected according to the intended purpose. Examples of the ketone group that may have a substituent include-CO-groups, acetyl groups, propionyl groups, and the like.

Note that, each of the ketone groups does not include a —CO— group included in a carboxyl group or aldehyde group.

The thioether group that may have a substituent is not particularly limited, and may be appropriately selected according to the intended purpose. Examples of the thioether group that may have a substituent include thiophenyl groups and the like.

The above-mentioned other functional groups are not particularly limited, and may be appropriately selected according to the intended purpose. Examples of the above-mentioned other functional groups include: hydroxyl groups; C1-C10 alkyl groups; phenyl groups; naphthyl groups; carboxyl groups; aldehyde groups; the above-mentioned ester group, amide group, ether group, ketone group, thioether group, and amino group different from the (i) amino group, all of which may have a substituent; and the like.

The hydrocarbon chain may be saturated hydrocarbon or unsaturated hydrocarbon, and may be of a straight chain, of a branched chain, or part of a cyclic structure.

A carbon atom in the hydrocarbon chain may be a primary carbon atom, a secondary carbon atom, or a tertiary carbon atom, and may be a carbon atom in a cyclic compound, such as an aromatic ring.

The (a) drug is not particularly limited, and may be appropriately selected according to the intended purpose. Examples of the therapeutic area of the (a) drug include local anesthetics, adrenergic receptor agonists, adrenergic receptor antagonists, acetylcholinesterase inhibitors, antipsychotics, serotonin receptor agonists, serotonin receptor antagonists, anti-allergic agents, ADHD drugs, hypnotics and sedatives, endocrine regulators, endothelin receptor antagonists, Parkinson's drugs, phosphodiesterase inhibitors, drugs for chronic pain, and the like.

Among the above-listed examples, local anesthetics, adrenergic receptor agonists, acetylcholinesterase inhibitors, antipsychotics, or serotonin receptor agonists are preferred, and local anesthetics are more preferred.

The local anesthetics are not particularly limited, and may be appropriately selected according to the intended purpose. Examples of the local anesthetics include tetracaine, lidocaine, prilocaine, procaine, chloroprocaine, mepivacaine, ropivacaine, bupivacaine, levobupivacaine, dibucaine, etidocaine, pharmaceutically acceptable salts of the foregoing substances, and the like.

Among the above-listed examples, tetracaine, lidocaine, prilocaine, or a pharmaceutically acceptable salt of any of the foregoing substances is preferred.

The adrenergic receptor agonists are not particularly limited, and may be appropriately selected according to the intended purpose. Examples of the adrenergic receptor agonists include naphazoline, mirabegron, vibegron, pharmaceutically acceptable salts of the foregoing substances, and the like.

The adrenergic receptor antagonists are not particularly limited, and may be appropriately selected according to the intended purpose. Examples of the adrenergic receptor antagonists include carvedilol, prazosin, phentolamine, doxazosin, phenoxybenzamine, yohimbine, propranolol, metoprolol, atenolol, bisoprolol, esmolol, carteolol, [symbol] , tamsulosin, pharmaceutically acceptable salts of the foregoing substances, and the like.

Among the above-listed examples, carvedilol, prazosin, phentolamine, doxazosin, phenoxybenzamine, yohimbine, propranolol, metoprolol, atenolol, bisoprolol, or a pharmaceutically acceptable salt of any of the foregoing substances is preferred.

The acetylcholinesterase inhibitors are not particularly limited, and may be appropriately selected according to the intended purpose. Examples of the acetylcholinesterase inhibitors include pyridostigmine, neostigmine, distigmine, galantamine, donepezil, tacrine, huperzine A, pharmaceutically acceptable salts of the foregoing substances, and the like.

The antipsychotics are not particularly limited, and may be appropriately selected according to the intended purpose. Examples of the antipsychotics include risperidone, quetiapine, perospirone, olanzapine, aripiprazole, clozapine, paliperidone, lurasidone, brexpiprazole, blonanserin, amoxapine, imipramine, oxypertine, clomipramine, spiperone, tiapride, timiperone, trimipramine, propericiazine, perphenazine, [symbol] , mianserin, mirtazapine, milnacipran, lofepramine, zotepine, paroxetine, bromperidol, pharmaceutically acceptable salts of the foregoing substances, and the like.

Among the above-listed examples, risperidone, quetiapine, perospirone, olanzapine, aripiprazole, clozapine, paliperidone, lurasidone, brexpiprazole, blonanserin, amoxapine, imipramine, oxypertine, clomipramine, spiperone, tiapride, timiperone, trimipramine, propericiazine, perphenazine, perospirone, mianserin, mirtazapine, milnacipran, lofepramine, or a pharmaceutically acceptable salt of any of the foregoing substances is preferred.

The serotonin receptor agonists are not particularly limited, and may be appropriately selected according to the intended purpose. Examples of the serotonin receptor agonists include tandospirone, buspirone, lysergic acid diethylamide, pharmaceutically acceptable salts of the foregoing substances, and the like.

The serotonin receptor antagonists are not particularly limited, and may be appropriately selected according to the intended purpose. Examples of the serotonin receptor antagonists include spiperone, ergotamine, ketanserin, ondansetron, ramosetron, clozapine, azasetron, granisetron, sarpogrelate, dimetotiazine, palonosetron, pharmaceutically acceptable salts of the foregoing substances, and the like.

The anti-allergic agents are not particularly limited, and may be appropriately selected according to the intended purpose. Examples of the anti-allergic agents include azelastine, emedastine, cetrizine, ibudilast, ebastine, diphenhydramine, pharmaceutically acceptable salts of the foregoing substances, and the like.

Among the above-listed examples, azelastine, emedastine, cetrizine, or a pharmaceutically acceptable salt of any of the foregoing substances is preferred.

The ADHD drugs are not particularly limited, and may be appropriately selected according to the intended purpose. Examples of the ADHD drugs include atomoxetine, guanfacine, pharmaceutically acceptable salts of the foregoing substances, and the like.

The hypnotics and sedatives are not particularly limited, and may be appropriately selected according to the intended purpose. Examples of the hypnotics and sedatives include amobarbital, alprazolam, zopiclone, eszopiclone, estazolam, oxazolam, quazepam, clotiazepam, suvorexant, zolpidem, triazolam, haloxazolam, phenobarbital, flunitrazepam, flurazepam, brotizolam, medazepam, lemborexant, lorazepam, lormetazepam, pharmaceutically acceptable salts of the foregoing substances, and the like.

The endocrine regulators are not particularly limited, and may be appropriately selected according to the intended purpose. Examples of the endocrine regulators include taltirelin, relugolix, clomifene, pharmaceutically acceptable salts of the foregoing substances, and the like.

Among the above-listed examples, taltirelin, relugolix, or a pharmaceutically acceptable salt of any of the foregoing substances is preferred.

The endothelin receptor antagonists are not particularly limited, and may be appropriately selected according to the intended purpose. Examples of the endothelin receptor antagonists include ambrisentan, bosentan, macitentan, pharmaceutically acceptable salts of the foregoing substances, and the like.

The Parkinson's drugs are not particularly limited, and may be appropriately selected according to the intended purpose. Examples of the Parkinson's drugs include istradefylline, opicapone, cabergoline, safinamide, talipexole, pramipexole, pergolide, rotigotine, pharmaceutically acceptable salts of the foregoing substances, and the like.

Among the above-listed examples, istradefylline, opicapone, cabergoline, safinamide, talipexole, pramipexole, or a pharmaceutically acceptable salt of any of the foregoing substances is preferred.

The phosphodiesterase inhibitors are not particularly limited, and may be appropriately selected according to the intended purpose. Examples of the phosphodiesterase inhibitors include sildenafil, tadalafil, vardenafil, pharmaceutically acceptable salts of the foregoing substances, and the like.

The drugs for chronic pain are not particularly limited, and may be appropriately selected according to the intended purpose. Examples of the drugs for chronic pain include fentanyl, pharmaceutically acceptable salts of the foregoing substance, and the like.

An amount of the (a) drug in the adhesive layer, i.e., a proportion of the adhesive layer to 100% by mass of a total amount of constituent components of the adhesive layer, is not particularly limited, and may be appropriately selected according to the intended purpose. In order to resin the (a) homogeneously in the adhesive layer without being precipitated, the lower limit of the amount of the (a) drug is preferably 0.5% by mass or greater, more preferably 1% by mass or greater, yet more preferably 1.5% by mass or greater, and particularly preferably 2% by mass or greater. The upper limit of the amount of the (a) drug is preferably 30% by mass or less, more preferably 25% by mass or less, yet more preferably 20% by mass or less, and particularly preferably 10% by mass or less.

### (b) Thermoplastic Elastomer

The thermoplastic elastomer is an elastomer exhibiting thermoplasticity such that the elastomer is softened to exhibit fluidity when heat is applied, and is returned back to an rubber-like elastic body when cooled. As the thermoplastic elastomer, various thermoplastic elastomers, such as urethane elastomers, acryl elastomers, styrene elastomers, olefin, silicone elastomers, and the like, have been known.

The above-mentioned various elastomers include derivatives thereof.

Among the above-listed examples, a thermoplastic elastomer of a styrene elastomer is more preferred, and a thermoplastic elastomer including a styrene-based block copolymer is yet more preferred because a large amount of a liquid component can be added.

The styrene-based block copolymer is not particularly limited, and may be appropriately selected in accordance with the intended purpose. Examples of the styrene-based block copolymer include a styrene-butadiene block copolymer, a styrene-butadiene-styrene block copolymer, a styrene-isoprene block copolymer, a styrene-isoprene-styrene block copolymer, a styrene-ethylene/butylene block copolymer, a styrene-ethylene/butylene-styrene block copolymer, a styrene-ethylene/propylene block copolymer, a styrene-ethylene/propylene-styrene block copolymer, a styrene-isobutylene block copolymer, a styrene-isobutylene-styrene block copolymer, and the like.

The term “ethylene/butylene” means a copolymer block of ethylene and butylene, and the term “ethylene/propylene” means a copolymer block of ethylene and propylene.

The above-listed styrene-based block copolymers may be used alone or in combination.

Among the above-listed styrene-based block copolymers, one, or two or more selected from the group consisting of a styrene-isoprene-styrene block copolymer and a styrene-isoprene block copolymer are particularly preferably used for achieving both adequate adhesion on skin and minimizing a residual adhesive owing to improvement in aggregation force of the adhesive layer, and considering availability of a product for patches and easiness of handling. The styrene-based block copolymer is most preferably a mixture of a styrene-isoprene-styrene block copolymer and a styrene-isoprene block copolymer.

When the mixture of the styrene-isoprene-styrene block copolymer and the styrene-isoprene block copolymer is used as the styrene-based block copolymer, the upper limit of an amount of the styrene-isoprene block copolymer in the mixture is not particularly limited, and may be appropriately selected according to the intended purpose. The upper limit is preferably 80% by mass or less for assuring adequate adhesion.

When the mixture of the styrene-isoprene-styrene block copolymer and the styrene-isoprene block copolymer is used as the styrene-based block copolymer, the lower limit of an amount of the styrene-isoprene block copolymer is not particularly limited, and may be appropriately selected according to the intended purpose. The lower limit is preferably 15% by mass or greater, more preferably 20% by mass or greater, yet more preferably 30% by mass or greater, particularly preferably 408 by mass or greater, and most preferably 50% by mass or greater for assuring adequate aggregation force.

The styrene content of the styrene-isoprene-styrene block copolymer is not particularly limited, and may be appropriately selected according to the intended purpose. The lower limit of the styrene content is preferably 5% by mass or greater, more preferably 10% by mass or greater. The upper limit is preferably 60% by mass or less, more preferably 50% by mass or less. Moreover, a weight average molecular weight of the styrene-isoprene-styrene block copolymer as measured by gel permeation chromatography (GPC) is not particularly limited, and may be appropriately selected according to the intended purpose. The weight average molecular weight is preferably 20,000 or greater and 500,000 or less, and more preferably 30,000 or greater and 300,000 or less.

Moreover, the styrene content of the styrene-isoprene block copolymer is not particularly limited, and may be appropriately selected according to the intended purpose. The lower limit of the styrene content is preferably 5% by mass or greater, more preferably 10% by mass or greater. The upper limit is preferably 60% by mass or less, more preferably 50% by mass or less. Moreover, a weight average molecular weight of the styrene-isoprene block copolymer as measured by gel permeation chromatography (GPC) is not particularly limited, and may be appropriately selected according to the intended purpose. The weight average molecular weight is preferably 10,000 or grater and 500,000 or less, and more preferably 20,000 or greater and 300,000 or less.

The styrene-isoprene-styrene block copolymer and the styrene-isoprene block copolymer may be each produced according to any methods available in the related art. Moreover, commercial products satisfying the above-described properties may be used as each of the styrene-isoprene-styrene block copolymer and the styrene-isoprene block copolymer. Moreover, a mixture of the styrene-isoprene-styrene block copolymer and the styrene-isoprene block copolymer is also commercially available. Commercial products of the mixture in which the styrene-isoprene-styrene block copolymer satisfying the above-described properties and the styrene-isoprene block copolymer satisfying the above-described properties are mixed at the above-mentioned mixing ratio can be suitably used.

Examples of the commercial products of the mixture include: “KRATON® D1111,” “KRATON® D1163,” “KRATON® D1113,” and “KRATON® D1119,” produced by KRATON POLYMERS; “JSR SIS® 5229,” “JSR SIS® 5002,” “JSR SIS® 5403,” and “JSR SIS® 5505,” produced by JSR Corporation; “Quintac® 3421,” “Quintac® 3433N,” “Quintac® 3520,” “Quintac® 3450,” and “Quintac 3270,” produced by Zeon Corporation; and the like.

Among the above-listed examples, “KRATON® D1163,” “KRATON® D1113,” “JSR SIS® 5403,” “JSR SIS® 5505,” “Quintac® 3433N,” and “Quintac® 3520” are preferred, and “JSR SIS® 5505” and “Quintac® 3520” are more preferred in view of a blending ratio between the triblock copolymer and the diblock copolymer and melt viscosity. The above-listed styrene-based block copolymers are each the mixture of the styrene-isoprene-styrene block copolymer and the styrene-isoprene block copolymer, where an amount of the styrene-isoprene block copolymer in the mixture is 50% by mass or greater.

An amount of the (b) thermoplastic elastomer in the adhesive layer, i.e., the proportion of the (b) thermoplastic elastomer to 100% by mass of a total amount of constituent components of the adhesive layer, is not particularly limited, and may be appropriately selected according to the intended purpose. In view of shape retention of the adhesive layer and adhesion on skin, the lower limit of the amount of the (b) thermoplastic elastomer is preferably 10% by mass or greater, more preferably 15% by mass or greater, and yet more preferably 20% by mass or greater, and the upper limit of the amount of the (b) thermoplastic elastomer is preferably 60% by mass or less, more preferably 55% by mass or less, yet more preferably 50% by mass or less.

(c) Non-Volatile Hydrocarbon Oil Having Kinematic Viscosity of 70 mm²/s or less at 40° C.

The (c) non-volatile hydrocarbon oil can be used as a plasticizer.

The (c) non-volatile hydrocarbon oil is not particularly limited, and may be appropriately selected according to the intended purpose. Examples of the (c) non-volatile hydrocarbon oil include liquid paraffin, squalene, squalane, and the like. The above-listed examples may be used alone or in combination.

Among the above-listed examples, liquid paraffin or squalene is preferred for suppressing delamination.

The liquid paraffin is colorless and odorless, and a mixture of fluid saturated hydrocarbons. As the liquid paraffin and light liquid paraffin, liquid paraffin and light liquid paraffin each meeting the standard specified by Japanese Pharmacopoeia, US Pharmacopoeia, etc. are suitably used.

The upper limit of the kinematic viscosity of the (c) non-volatile hydrocarbon oil at 40° C. is not particularly limited, as long as the upper limit of the kinematic viscosity is 70 mm²/s or less. In view of suppression of delamination, the upper limit is preferably 60 mm²/s or less, more preferably 50 mm²/s or less, yet more preferably 40 mm²/s, yet further more preferably 30 mm²/s or less, particularly preferably 20 mm²/s or less, and most preferably 10 mm²/s.

The lower limit of the kinematic viscosity of the (c) non-volatile hydrocarbon oil at 40° C. is not particularly limited. In order to assure adequate aggregation force of the adhesive layer, the lower limit is preferably 1 mm²/s or greater, more preferably 2 mm²/s or greater, yet more preferably 3 mm²/s or greater, and particularly preferably 5 mm²/s or greater.

The “kinematic viscosity” is a value obtained by converting a viscosity (mPa·s) measured according to “Method II Viscosity measurement by rotational viscometer (2.12 Single cylinder-type rotational viscometer (Brookfield type viscometer)” in “2.53 Viscosity Determination” of General Tests in “Japanese Pharmacopoeia 17th Edition” into a kinematic viscosity.

Examples of commercial products of the (c) non-volatile hydrocarbon oil include “HICALL M-52” produced by KANEDA Co., Ltd., “HICALL M-72” produced by KANEDA Co., Ltd., “HICALL M-172” produced by KANEDA Co., Ltd., “Kaydol” produced by Sonneborn LLC, “Rudol” produced by Sonneborn LLC, “Ervol” produced by Sonneborn LLC, “Benol” produced by Sonneborn LLC, “Blandol” produced by Sonneborn LLC, “Carnation” produced by Sonneborn LLC, “Klearol” produced by Sonneborn LLC, “Lytol” produced by Sonneborn LLC, “SQUALANE” produced by KISHIMOTO SPECIAL LIVER OIL CO., LTD., and the like.

Among the above-listed examples, “HICALL M-52” produced by KANEDA Co., Ltd., “HICALL M-72” produced by KANEDA Co., Ltd., “HICALL M-172” produced by KANEDA Co., Ltd., “Carnation” produced by Sonneborn LLC, or “Klearol” produced by Sonneborn LLC is preferred.

The upper limit of an amount of the (c) non-volatile hydrocarbon oil is not particularly limited, except that the upper limit of the amount of the (c) non-volatile hydrocarbon oil is 230 parts by mass or less relative to 100 parts by mass of the (b) thermoplastic elastomer. In order to assure aggregation force of the adhesive layer, the upper limit of the amount of the (c) non-volatile hydrocarbon oil is preferably 220 parts by mass or less, more preferably 210 parts by mass or less, and yet more preferably 200 parts by mass or less, relative to 100 parts by mass of the (b) thermoplastic elastomer.

The lower limit of the amount of the (c) non-volatile hydrocarbon oil is not particularly limited. In order to assure adhesion of the adhesive layer, the lower limit of the amount of the (c) non-volatile hydrocarbon oil is preferably 30 parts by mass or greater, more preferably 40 parts by mass or greater, yet more preferably 50 parts by mass or greater, yet further more preferably 60 parts by mass or greater, particularly preferably 70 parts by mass or greater, and most preferably 80 parts by mass or greater, relative to 100 parts by mass of the (b) thermoplastic elastomer.

The upper limit of an amount of the (c) non-volatile hydrocarbon oil in the adhesive layer, i.e., the upper limit of the proportion of the (c) non-volatile hydrocarbon oil to 100% by mass of a total amount of constituent components of the adhesive layer, is not particularly limited, and may be appropriately selected according to the intended purpose. In order to assure aggregation force of the adhesive layer, the upper limit of the amount of the (c) non-volatile hydrocarbon oil is preferably 60% by mass or less, more preferably 55% by mass or less, and yet more preferably 50% by mass or less.

The lower limit of the amount of the (c) non-volatile hydrocarbon oil in the adhesive layer, i.e., the lower limit of the proportion of the (c) non-volatile hydrocarbon oil to 100% by mass of a total amount of constituent components of the adhesive layer, is not particularly limited, and may be appropriately selected according to the intended purpose. In order to assure adequate adhesion of the adhesive layer, the lower limit of the amount of the (c) non-volatile hydrocarbon oil is preferably 10% by mass or greater, more preferably 15% by mass or greater, yet more preferably 20% by mass or greater, yet further more preferably 25% by mass or greater, particularly preferably 30% by mass or greater, and most preferably 35% by mass or greater.

### —Other Components—

The above-mentioned other components are not particularly limited, and may be appropriately selected according to the intended purpose. Examples of the above-mentioned other components include (d) a solubilizer/absorption promoter, (e) a [symbol] [symbol] , (f) a softening agent, (g) an adsorbent, (h) a tackifier, (i) an antioxidant, and the like.

### (d) Solubilizer/Absorption Promoter

The (d) solubilizer/absorption promoter is not particularly limited, and may be appropriately selected according to the intended purpose. Examples of the (d) solubilizer/absorption promoter include: straight-chain saturated aliphatic alcohols, such as lauryl alcohol, myristyl alcohol, cetanol, cetostearyl alcohol, stearyl alcohol, behenyl alcohol, and the like; branched-chain saturated aliphatic alcohols, such as isostearyl alcohol, hexyldecanol, octyldodecanol, and the like; unsaturated aliphatic alcohols, such as oleyl alcohol, geraniol, and the like; polyhydric alcohols, such as ethylene glycol, propylene glycol, glycerin, 1,3-butanediol, dipropylene glycol, triethylene glycol, 2-ethyl-1,3-hexanediol, 1,2,6-hexanetriol, and the like; polyether-based alcohols, such as polyethylene glycol 200, polyethylene glycol 400, polyoxyethylene (2) ethyl ether (diethylene glycol monoethyl ether), polyoxyethylene (2) lauryl ether, polyoxyethylene (4) lauryl ether, polyoxyethylene (9) lauryl ether, polyoxyethylene (2) cetyl ether, polyoxyethylene (2) stearyl ether, monostearic acid ethylene glycol, and the like; propylene glycol monofatty acid ester, such as propylene glycol monocaproate, propylene glycol monocaprylate, propylene glycol monocaprate, propylene glycol monolaurate, propylene glycol monomyristate, propylene glycol monostearate, propylene glycol monopalmitostearate, and the like; aromatic alcohols, such as benzyl alcohol, phenylethyl alcohol, phenoxyethanol, thymol, eugenol, 2-naphthol, vanillin, and the like; cyclic alcohols, such as maltol, ethyl maltol, and the like; alkyl lactate compounds, such as ethyl lactate, lauryl lactate, cetyl lactate, and the like; salicylic acid derivatives, such as ethylene glycol salicylate, phenyl salicylate, methyl salicylate, and the like; citric acid derivatives, such as triethyl citrate, tributyl citrate, and the like; terpenes, such as α-termineol, D-borneol, DL-borneol, L-menthol, DL-menthol, and the like; sorbitans, such as sorbitan monooleate, sorbitan monostearate, sorbitan tristearate, sorbitan monolaurate, sorbitan monopalmitate, sorbitan sesquioleate, sorbitan trioleate, polyoxyethylene sorbitan monolaurate, polyoxyethylene sorbitan monostearate, polyoxyethylene sorbitan trioleate, polyoxyethylene sorbitan tetraoleate, and the like; glycerin derivatives, such as glyceryl monooleate, glyceryl monostearate, glyceryl monomyristate, diglyceryl monooleate, decaglyceryl laurate, α-monoisostearyl glyceryl ether, diglyceryl monoisostearate, diglyceryl monostearate, polyoxyethylene glyceryl monostearate, polyoxyethylene glyceryl triisostearate, polyoxyethylene glyceryl cocoate, and the like; stearic acid derivatives, such as propylene glycol monostearate, ethylene glycol monostearate, and the like; esters between fatty acids and monovalent aliphatic alcohols, such as isoamyl isovalerate, isostearyl palmitate, cetyl 2-ethylhexanoate (cetyl isooctanoate), ethyl oleate, decyl oleate, isopropyl palmitate, cetyl palmitate, isopropyl myristate, cetyl myristate, myristyl myristate, batyl monostearate, hexyl laurate, methyl laurate, isopropyl linoleate, ethyl linoleate, cocoyl caprylocaprate, and the like; diesters, such as diisopropyl adipate, diisobutyl adipate, diisopropyl sebacate, diethyl sebacate, and the like; propylene glycol-based diesters, such as propylene glycol dicaprylate, propylene glycol dicaprate, propylene glycol dicaprylocaprate, propylene glycol diacetate, and the like; glycerin-based esters, such as triacetin, tricaprylin (glycerol trioctanoate), caprylic/capric triglyceride, glyceryl trioctanoate (triethylhexanoin), triglycerol diisostearate, medium-chain triglycerides, and the like; citric acid-based esters, such as triethyl O-acetylcitrate, tributyl O-acetylcitrate, and the like; cyclic carbonates, such as ethylene carbonate, propylene carbonate, maleic anhydride, and the like; aromatic esters, such as benzyl benzoate, benzyl acetate, diethyl phthalate, dibutyl phthalate, butyl phthalyl butyl glycolate, and the like; fatty acid glycerol macrogol esters, such as linoleoyl macrogol-6 glyceride, and the like; organic acid, such as lactic acid, caprylic acid, capric acid, isostearic acid, oleic acid, levulinic acid, and the like; and amide, such as crotamiton, N-methylpyrrolidone, and the like. The above-listed examples may be used alone or in combination.

Among the above-listed examples, oleyl alcohol, propylene glycol monocaprylate, diisopropyl adipate, linoleoyl macrogol-6 glyceride, capric acid, oleic acid, levulinic acid, crotamiton, or N-methylpyrrolidone is preferred in view of improvement in solubility of the drug and improvement in permeation through skin.

### (e) Salt Dissociation Agent

The (e) [symbol] is not particularly limited, and may be appropriately selected according to the intended purpose. Examples of the (e) salt dissociation agent include: amines, such as monoethanolamine, diethanolamine, triethanolamine, diisopropanolamine, triisopropanolamine, and the like; aminoalkyl-based polymers, such as aminoalkyl methacrylate copolymer E, aminoalkyl methacrylate copolymer RS, aminoalkyl methacrylate copolymer RL, and the like; and inorganic salts, such as sodium hydroxide, sodium carbonate, and the like. The above-listed examples may be used alone or in combination.

Among the above-listed examples, diethanolamine or diisopropanolamine is preferred in view of improvement in solubility of the components in the adhesive layer.

### (f) Softening Agent

The (f) softening agent is not particularly limited, and may be appropriately selected according to the intended purpose. Examples of the (f) softening agent include polybutene, polyisobutylene, and the like.

### (g) Adsorbent

The (g) adsorbent is not particularly limited, and may be appropriately selected according to the intended purpose. Examples of the (g) adsorbent include: silicon compounds, such as silicic anhydride, light anhydrous silicic acid, silicic acid hydrate, and the like; cellulose derivatives, such as ethyl cellulose, methyl cellulose, hydroxypropyl cellulose, hydroxypropyl methyl cellulose, and the like; water-soluble polymers, such as polyvinyl alcohol and the like; aluminum compounds, such as dried aluminum hydroxide gel, hydrous aluminum silicate, and the like; kaolin; titanium oxide; and the like. The above-listed examples may be used alone or in combination.

Among the above-listed examples, light anhydrous silicic acid is preferred in view of homogeneous dispersibility.

### (h) Tackifier

The (h) tackifier is not particularly limited, and may be appropriately selected according to the intended purpose. Examples of the (h) tackifier include: rosin-based resins, such as rosin esters and the like; polyterpene-based resins; coumarone-indene resins; petroleum resins; terpene resins; terpene-phenol resins; alicyclic saturated hydrocarbon resins; and the like. The above-listed examples may be used alone or in combination.

Among the above-listed examples, a terpene resin or an alicyclic saturated hydrocarbon resin is preferred in view of improvement in drug penetration through skin.

### (i) Antioxidant

The (i) antioxidant is not particularly limited, and may be appropriately selected according to the intended purpose. Examples of the (i) antioxidant include: phenol-based antioxidants, such as dibutylhydroxytoluene, butylhydroxyanisole, propyl gallate, and the like; sulfur-containing antioxidants, such as 2-mercaptobenzimidazole, α-thioglycerol, and the like; and vitamins, such as ascorbic acid, riboflavin, hesperidin, tocopherol, and the like.

An average thickness of the adhesive layer when dried is not particularly limited, and may be appropriately selected according to the intended purpose. In view of improvement in adhesion of the adhesive layer, the lower limit of the average thickness of the adhesive layer is preferably 50 μm or greater, more preferably 100 μm or greater, and yet more preferably 200 μm or greater. In view of production efficiency, the upper limit of the average thickness of the adhesive layer is preferably 1,000 μm or less, more preferably 500 μm or less.

### <Other Elements>

The above-mentioned other elements are not particularly limited, and may be appropriately selected in accordance with the intended purpose. Examples of the above-mentioned other elements include a support, a release liner, and the like. In other words, the patch of the present invention may include a support, an adhesive layer, and a release liner stacked in this order.

The support is not particularly limited, and may be appropriately selected in accordance with the intended purpose. Examples of the support include supports generally used for adhesive sheets to adhere to skin or for transdermal drugs.

A material of the support is not particularly limited, and may be appropriately selected in accordance with the intended purpose. Examples of the material include polyesters (e.g., polyethylene terephthalate and the like), polyolefins (e.g., polyethylene, polypropylene, and the like), polyurethanes, ethylene-vinyl acetate copolymers, polyvinyl chloride, and the like.

A structure of the support may be a single-layer structure or a multiple-layer structure. Moreover, the support may be of knitted fabrics, woven fabrics, nonwoven fabrics, a film, foam, a porous body, a network structure, a sheet, or a flat plate.

In order to minimize accumulation of static electricity within the support, an antistatic agent may be added to the woven fabrics, nonwoven fabrics, film, or the like, constituting the support. Moreover, nonwoven fabrics or woven fabrics, or a laminate of a film with nonwoven fabrics and woven fabrics may be used as the support to assure excellent anchoring to an adhesive layer.

An average thickness of the support is not particularly limited, and may be appropriately selected according to the intended purpose. For a film serving as the support, the lower limit of the average thickness of the support is preferably 10 μm or greater, more preferably 15 μm or greater, and the upper limit of the average thickness of the support is preferably 100 μm or less, more preferably 50 μm or less. For a porous sheet serving as the support, such as a woven fabric support, a nonwoven fabric support, a foam support, and the like, the lower limit of the average thickness of the support is preferably 50 μm or greater, more preferably 100 μm or greater, and the upper limit of the average thickness of the support is preferably 2,000 μm or less, more preferably 1,000 μm or less.

The release liner is not particularly limited, and may be appropriately selected in accordance with the intended purpose. Examples of the release liner include: resin films, such as glassine, polyolefins (e.g., polyethylene, polypropylene, and the like), polyesters (e.g., polyethylene terephthalate and the like), polystyrenes, and the like; aluminum films; polyethylene foam films or polypropylene foam films; and laminates each including two or more of the foregoing liners. Moreover, the release liner subjected to silicone processing, fluororesin processing, embossing, hydrophilic processing, hydrophobic processing, or the like may be used.

An average thickness of the release liner is not particularly limited, and may be appropriately selected according to the intended purpose. The lower limit of the average thickness of the release liner is preferably 10 μm or greater, more preferably 15 μm or greater, and the upper limit of the average thickness of the release liner is preferably 200 μm or less, more preferably 150 μm or less.

### Production Method of Patch

The production method of the patch includes a mixing step of mixing (a) a drug, (b) a thermoplastic elastomer, and (c) non-volatile hydrocarbon oil having a kinematic viscosity of 70 mm²/s or less at 40° C., and may further include other steps.

The (a) drug, the (b) thermoplastic elastomer, and the (c) non-volatile hydrocarbon oil having a kinematic viscosity of 70 mm²/s or less at 40° C.

### <Mixing Step>

The mixing step is not particularly limited, and may be appropriately selected according to the intended purpose. The mixing step is preferably a method where mixing is performed in the presence of a volatile solvent.

The volatile solvent is not particularly limited, and may be appropriately selected according to the intended purpose. Examples of the volatile solvent include: aromatic hydrocarbons, such as toluene and the like; alicyclic hydrocarbons, such as cyclohexane, methyl cyclohexane, and the like; aliphatic hydrocarbons, such as hexane, heptane, and the like; ethers, such as tetrahydrofuran, diethyl ether, t-butyl methyl ether, and the like; ketones, such as acetone, methyl ethyl ketone, methyl isobutyl ketone, and the like; alcohols, such as ethanol, propanol, butanol, and the like; and acetic acid esters, such as ethyl acetate, propyl acetate, isopropyl acetate, butyl acetate, isobutyl acetate, and the like. The above-listed examples may be used alone or in combination.

Among above-listed examples, use of a single solvent or a mixture of two or more solvents selected from aromatic hydrocarbons (e.g., toluene and the like), alicyclic hydrocarbons (e.g., cyclohexane, methyl cyclohexane, and the like), and aliphatic hydrocarbons (e.g., hexane, heptane, and the like) is preferred in view of desired solubility of constituent components of the adhesive layer.

### <Other Steps>

The above-mentioned other steps are not particularly limited, and may be appropriately selected according to the intended purpose. Examples of the above-mentioned other steps include a coating step after the mixing step, a laminating step of an adhesive layer and a release liner, a laminating step of an adhesive layer and a support, and the like.

The support, the adhesive layer, and the release liner are as described above.

### —Coating Step After the Mixing Step—

Coating of an adhesive layer-forming coating liquid obtained in the mixing step is not particularly limited, and may be appropriately selected according to the intended purpose. For example, coating of the coating liquid may be performed on the release liner or the support by a commonly used coater, such as a roll coater, a die coater, a gravure roll coater, a reverse roll coater, a kiss roll coater, a dip roll coater, a bar coater, a knife coater, a spray coater, and the like. Moreover, the coating liquid is preferably dried with heat, for example, at a temperature of 40° C. or higher and 150° C. or lower. A drying temperature, drying duration, and drying system may be adjusted according to a solvent used and an amount of the solvent used.

### —Laminating Step of Adhesive Layer and Release Liner—

The laminating step of the adhesive layer and the release liner is not particularly limited, and may be appropriately selected according to the intended purpose. Examples of the laminating step of the adhesive layer and the release liner include: a step including spreading the adhesive layer-forming coating liquid on a release liner, and drying the solvent in the coating liquid to laminate an adhesive layer on a surface of the release liner (spreading and drying step); a step including, after the laminating step of the adhesive layer and the support, pressure-bonding the release liner onto the adhesive layer to laminate the release liner on the adhesive layer; and the like.

### —Laminating Step of Adhesive Layer and Support—

The laminating step of the support and the adhesive layer is not particularly limited, and may be appropriately selected according to the intended purpose. Examples of the laminating step of the support and the adhesive layer include: a step including spreading the adhesive layer-forming coating liquid on a support, and drying the solvent in the coating liquid to laminate an adhesive layer on a surface of the support (spreading and drying step); a step including, after the laminating step of the adhesive layer and the release liner, pressure-bonding the support onto the adhesive layer to laminate the support on the adhesive layer; and the like.

### (Anti-Delamination Agent)

The anti-delamination agent includes (a) a drug, (b) a thermoplastic elastomer, and (c) non-volatile hydrocarbon oil having a kinematic viscosity of 70 mm²/s or less at 40° C. The anti-delamination agent may further include other components.

The (a) drug, the (b) thermoplastic elastomer, the (c) non-volatile hydrocarbon oil having a kinematic viscosity of 70 mm²/s or less at 40° C., and the above-mentioned other components are as described above.

The anti-delamination is to suppress a phenomenon where the support is detached from the adhesive layer of the patch, and is also referred to as suppression of delamination.

### (Method for Suppressing Delamination)

The method for suppressing delamination is a method where the anti-delamination agent is used for a patch.

The anti-delamination agent is as described above.

### EXAMPLES

Examples of the present invention will be described hereinafter, but Examples shall not be construed as limiting the scope of the present invention in any way.

### <Kinematic Viscosity of Non-Volatile Hydrocarbon Oil>

Kinematic viscosities of non-volatile hydrocarbon oil used in Examples are presented in Table 1.

TABLE 1

| | Viscosity | Kinematic |
|---|---|---|
| Grade of non-volatile | measuring | viscosity |
| hydrocarbon oil | temperature (° C.) | (mm²/s) |
| HICALL M-52 | 37.8 | 5.8-8.9 |
| (light liquid paraffin) | | |
| HICALL M-72 | 37.8 | 11.7-15.7 |
| (light liquid paraffin) | | |
| HICALL M-172 | 37.8 | 33.0-36.9 |
| (light liquid paraffin) | | |
| Kaydol (liquid paraffin) | 40 | 66.3-68.7 |
| Hydrobrite 550PO | 40 | 100-125 |
| (liquid paraffin) | | |
| Hydrobrite HV | 40 | 247 |
| (liquid paraffin) | | |
| SQUALANE | 40 | 20.5 |

### Example 1

Each of constituent components of an adhesive layer was weighed according to the composition of Example 1 presented in Table 2. Note that, a numerical value of each component in Table 2 is expressed by % by mass.

First, tetracaine hydrochloride, diethanolamine (“Diethanolamine” produced by Mitsui Chemicals, Inc.), propylene glycol monocaprylate (“Capryol 90” produced by Gattefosse), oleyl alcohol (“NOVOL HC-LQ-JP” produced by CRODA), diisopropyl adipate (“DID” produced by Nikko Chemicals Co., Ltd.), and light anhydrous silicic acid (“SYLYSIA 320” produced by FUJI SILYSIA CHEMICAL LTD.) were mixed and stirred at room temperature to obtain a mixture A.

A mixture of a styrene-isoprene-styrene block copolymer and a styrene-isoprene block copolymer (“Quintac 3520 SIS/SI ratio=22/78”, produced by Zeon Corporation) and a terpene resin (“YS Resin PX1150N” produced by YASUHARA CHEMICAL CO., LTD.) were mixed and stirred in a toluene (internal temperature: 20° C. to 40° C.) to dissolve the mixture and the terpene resin. To the resulting solution, the mixture A was added, followed by mixing and stirring at room temperature. To the resulting mixture, light liquid paraffin (“HICALL M-52” produced by KANEDA Co., Ltd.) was further added, followed by mixing stirring at room temperature, to prepare an adhesive layer-forming coating liquid.

The coating liquid was applied onto a silicone-processed polyethylene terephthalate (PET) film (release liner, “FILMBYNA 75E-0010 BD” produced by FUJIMORI KOGYO CO., LTD.) in a manner that an average thickness of an adhesive layer after drying was to be approximately 350 μm. The applied coating liquid was dried in an oven of 80° C. for 30 minutes, followed by laminating a PET film (support) on a surface of the resulting adhesive layer, to thereby obtain a patch.

The presence or absence of delamination in the patch was evaluated according to the following evaluation criteria. The result is presented in Table 2.

### <Evaluation Criteria>

Absence of delamination: When a finger was strongly pressed against the adhesive layer and released from the adhesive layer just after the production of the patch, the adhesive layer was not peeled from the support.
Presence of delamination: When a finger was strongly pressed against the adhesive layer and released from the adhesive layer just after the production of the patch, the adhesive layer was peeled from the support.

TABLE 2

| | | | | | | Comp. | Comp. | Comp. | Comp. |
|---|---|---|---|---|---|---|---|---|---|
| | | Ex. 1 | Ex. 2 | Ex. 3 | Ex. 4 | Ex. 1 | Ex. 2 | Ex. 3 | Ex. 4 |
| Drug | Tetracaine | 4.5 | 4.5 | 4.5 | 4.5 | 4.5 | 4.5 | 4.5 | |
| | hydrochloride | | | | | | | | |
| | Minodronic | | | | | | | | 4.5 |
| | acid | | | | | | | | |
| Base polymer | SIS (Quintac | 34.0 | 34.0 | 34.0 | 34.0 | 34.0 | 34.0 | 34.0 | 34.0 |
| | 3520) | | | | | | | | |
| Plasticizer | Light liquid | 40.8 | | | | | | | 40.8 |
| | paraffin | | | | | | | | |
| | (HICALL M-52) | | | | | | | | |
| | Light liquid | | 40.8 | | | | | | |
| | paraffin | | | | | | | | |
| | (HICALL M-72) | | | | | | | | |
| | Light liquid | | | 40.8 | | | | | |
| | paraffin | | | | | | | | |
| | (HICALL M-172) | | | | | | | | |
| | SQUALANE | | | | 40.8 | | | | |
| | Liquid | | | | | 40.8 | | | |
| | paraffin | | | | | | | | |
| | (Hydrobrite | | | | | | | | |
| | 550PO) | | | | | | | | |
| | Liquid | | | | | | 40.8 | 40.8 | |
| | paraffin | | | | | | | | |
| | (Hydrobrite | | | | | | | | |
| | HV) | | | | | | | | |
| Solubilizer/ | Propylene | 5.25 | 5.25 | 5.25 | 5.25 | 5.25 | 5.25 | 5.25 | 5.25 |
| absorption | glycol | | | | | | | | |
| promoter | monocaprylate | | | | | | | | |
| | Oleyl alcohol | 2.25 | 2.25 | 2.25 | 2.25 | 2.25 | 2.25 | 2.25 | 2.25 |
| | Diisopropyl | 1.50 | 1.50 | 1.50 | 1.50 | 1.50 | 1.50 | 1.50 | 1.50 |
| | adipate | | | | | | | | |
| Salt | Diethanol | 1.6 | 1.6 | 1.6 | 1.6 | 1.6 | 1.6 | 1.6 | 1.6 |
| dissociation | amine | | | | | | | | |
| agent | | | | | | | | | |
| Adsorbent | Light | 0.1 | 0.1 | 0.1 | 0.1 | 0.1 | 0.1 | 0.1 | 0.1 |
| | anhydrous | | | | | | | | |
| | silicic acid | | | | | | | | |
| | (SYLYSIA 320) | | | | | | | | |
| Tackifier | Terpene resin | 10.0 | 10.0 | 10.0 | 10.0 | 10.0 | 10.0 | 10.0 | 10.0 |

| | | | | | | | | |
|---|---|---|---|---|---|---|---|---|
| Support | PET | PET | PET | PET | PET | PET | Laminate | PET |
| | film | film | film | film | film | film | of PET | film |
| | | | | | | | film and | |
| | | | | | | | PET | |
| | | | | | | | nonwoven | |
| | | | | | | | fabric | |
| Delamination | Absent | Absent | Absent | Absent | Present | Present | Present | Present |

### Examples 2 to 4 and Comparative Examples 1 to 2

A patch was produced and the presence or absence of delamination was evaluated in the same manner as in Example 1, except that each of constituent components of an adhesive layer was weighed according to the composition presented in Table 2. The results are presented in Table 2.

### Comparative Example 3

A patch was produced and the presence or absence of delamination was evaluated in the same manner as in Comparative Example 2, except that a laminate of a PET film and a PET nonwoven fabric was used as the support. The result is presented in Table 2.

### Comparative Example 4

A patch was produced and the presence or absence of delamination was evaluated in the same manner as in Example 1, except that minodronic acid was used as the drug. The result is presented in Table 2.

It was found from the results presented in Table 2 that use of the plasticizer having the kinematic viscosity of 70 mm²/s or less at 40° C. (specifically, HICALL M-52, HICALL M-72, HICALL M-172, or SQUALANE) could suppress the delamination of the adhesive layer. Conversely, use of the plasticizer having the kinematic viscosity of 70 mm²/s or greater at 40° C. (specifically, Hydrobrite 550PO or Hydrobrite HV) resulted in delamination of the adhesive layer.

When the plasticizer having the kinematic viscosity of 70 mm²/s or greater at 40° C. was used, moreover, delamination occurred regardless of the support that was replaced. Therefore, it was found that delamination could not be suppressed by changing the support. When the minodronic acid including five hydroxyl groups was used as the drug, moreover, delamination occurred even with use of the plasticizer having the kinematic viscosity of 70 mm²/s or less at 40° C.

### Example 5

Each of constituent components of an adhesive layer was weighed according to the composition of Example 5 presented in Table 3. Note that, a numerical value of each component in Table 3 is expressed by % by mass.

First, tetracaine hydrochloride, diisopropanolamine (“diisopropanolamine” produced by MITSUI FINE CHEMICALS, INC.), propylene glycol monocaprylate (“Capryol 90” produced by Gattefosse), linoleoyl macrogol-6 glyceride (“Labrafil M 2125 CS” produced by Gattefosse), and diisopropyl adipate (“DID” produced by Nikko Chemicals Co., Ltd.) were mixed and stirred at room temperature to obtain a mixture A.

A mixture of a styrene-isoprene-styrene block copolymer and a styrene-isoprene block copolymer (“Quintac 3520 SIS/SI ratio=22/78” produced by Zeon Corporation), polybutene (“INDOPOL H-300” produced by INEOS Oligomers), and an alicyclic saturated hydrocarbon resin (“ARKON P-100” produced by ARAKAWA CHEMICAL INDUSTRIES, LTD.) were mixed and stirred in a toluene (internal temperature: 20° C. to 40° C.) to dissolve the mixture, the polybutene, and the alicyclic saturated hydrocarbon resin. To the resulting solution, the mixture A was added, followed by mixing and stirring at room temperature. To the resulting mixture, light liquid paraffin (“HICALL M-52” produced by KANEDA Co., Ltd.) was further added, followed by mixing stirring at room temperature, to prepare an adhesive layer-forming coating liquid.

The coating liquid was applied onto a silicone-processed polyethylene terephthalate (PET) film (release liner, “FILMBYNA 75E-0010 BD” produced by FUJIMORI KOGYO CO., LTD.) in a manner that an average thickness of an adhesive layer after drying was to be approximately 350 μm. The applied coating liquid was dried in an oven of 80° C. for 30 minutes, followed by laminating a PET film (support) on a surface of the resulting adhesive layer, to thereby obtain a patch.

The presence or absence of delamination in the patch was evaluated in the same manner as in Example 1. The results are presented in Table 3.

TABLE 3

| | | | Comp. | Comp. |
|---|---|---|---|---|
| | | Ex. 5 | Ex. 5 | Ex. 6 |
| Drug | Tetracaine | 4.0 | 4.0 | 4.0 |
| | hydrochloride | | | |
| Base polymer | SIS (Quintac 3520) | 21.0 | 21.0 | 21.0 |
| Plasticizer | Light liquid paraffin | 35.0 | | |
| | (HICALL M-52) | | | |
| | Liquid paraffin | | 35.0 | |
| | (Hydrobrite 550PO) | | | |
| | Liquid paraffin | | | 35.0 |
| | (Hydrobrite HV) | | | |
| Solubilizer/ | Propylene glycol | 4.67 | 4.67 | 4.67 |
| absorption | monocaprylate | | | |
| promotor | Linoleoyl macrogol-6 | 2.00 | 2.00 | 2.00 |
| | glyceride | | | |
| | Diisopropyl adipate | 1.33 | 1.33 | 1.33 |
| Salt | Diisopropanolamine | 2.2 | 2.2 | 2.2 |
| dissociation | | | | |
| agent | | | | |
| Softening agent | Polybutene | 15.0 | 15.0 | 15.0 |
| Tackifier | Alicyclic saturated | 14.8 | 14.8 | 14.8 |
| | hydrocarbon rein | | | |

| | | | |
|---|---|---|---|
| Support | PET film | PET film | PET film |
| Delamination | Absent | Present | Present |

### Comparative Examples 5 to 6

A patch was produced and the presence or absence of delamination was evaluated in the same manner as in Example 1-5, except that each of constituent components of an adhesive layer was weighed according to the composition presented in Table 3. The results are presented in Table 3.

It was found from the results presented in Table 3 that use of the plasticizer having the kinematic viscosity of 70 mm²/s or less at 40° C. (specifically, HICALL M-52) could suppress the delamination of the adhesive layer. Conversely, delamination of the patches of Comparative Examples 5 to 6 each including the plasticizer having the kinematic viscosity of 70 mm²/s or greater at 40° C. (specifically, Hydrobrite 550PO or Hydrobrite HV) occurred.

### Examples 6 to 8

A patch was produced and the presence or absence of delamination was evaluated in the same manner as in Example 1, except that each of constituent components of an adhesive layer was weighed according to the composition presented in Table 4. The results are presented in Table 4. Note that, a numerical value of each component in Table 4 is expressed by % by mass.

TABLE 4

| | | Ex. 6 | Ex. 7 | Ex. 8 |
|---|---|---|---|---|
| Drug | Lidocaine | 5.0 | 5.0 | 5.0 |
| | Prilocaine | 5.0 | 5.0 | 5.0 |
| Base polymer | SIS (Quintac 3520) | 44.0 | 44.0 | 44.0 |
| Plasticizer | Light liquid paraffin | 46.0 | 25.0 | |
| | (HICALL M-52) | | | |
| | Liquid paraffin | | 21.0 | 46.0 |
| | (KAYDOL) | | | |

| | | | |
|---|---|---|---|
| Support | PET film | PET film | PET film |
| Delamination | Absent | Absent | Absent |

It was found from the results presented in Table 4 that use of the plasticizer having the kinematic viscosity of 70 mm²/s or less at 40° C. (specifically, HICALL M-52 and KAYDOL) could suppress the delamination of the adhesive layer.

For example, embodiments of the present invention are as follows.
<1> A patch including:
   an adhesive layer including:
      (a) a drug;
      (b) a thermoplastic elastomer; and
      (c) non-volatile hydrocarbon oil having a kinematic viscosity of 70 mm²/s or less at 40° C., wherein
   the (a) drug includes
   (i) an amino group that may have a substituent, and
   (ii) at least one functional group selected from the group consisting of an ester group, an amide group, an ether group, a ketone group, a thioether group, and an amino group different from the (i) amino group, all of which may have a substituent, where
      the (a) drug has a structure, in which the (i) amino group, and the (ii) at least one functional group are bonded via a C1-C3 hydrocarbon chain, and
      the number of hydroxyl groups per molecule of the (a) drug is 4 or less, and
   an amount of the (c) non-volatile hydrocarbon oil is 230 parts by mass or less relative to 100 parts by mass of the (b) thermoplastic elastomer.
<2> The patch according to <1>,
   wherein the (a) drug includes
      (i) an amino group that may have a substituent, and
      (ii) at least one functional group selected from the group consisting of an ester group, an amide group, and an amino group different from the (i) amino group, all of which may have a substituent,
   where the (a) drug has a structure, in which the (i) amino group, and the (ii) at least one functional group are bonded via a C1 or C2 hydrocarbon chain.
<3> The patch according to <1> or <2>,
   wherein the (b) thermoplastic elastomer includes a styrene-based block copolymer.
<4> The patch according to <3>,
   wherein the styrene-based block copolymer includes a mixture of a styrene-isoprene-styrene block copolymer and a styrene-isoprene block copolymer.
<5> The patch according to any one of <1> o <4>,
wherein the patch is a matrix patch.
<6> A production method of the patch of any one of <1> to <5>, including:
   mixing
      the (a) drug;
      the (b) thermoplastic elastomer; and
      the (c) non-volatile hydrocarbon oil non-volatile hydrocarbon oil having a kinematic viscosity of 70 mm²/s or less at 40° C., wherein
   the (a) drug includes
   (i) an amino group that may have a substituent, and
   (ii) at least one functional group selected from the group consisting of an ester group, an amide group, an ether group, a ketone group, a thioether group, and an amino group different from the (i) amino group, all of which may have a substituent, where
      the (a) drug has a structure, in which the (i) amino group, and the (ii) at least one functional group are bonded via a C1-C3 hydrocarbon chain, and
      the number of hydroxyl groups per molecule of the (a) drug is 4 or less, and
   an amount of the (c) non-volatile hydrocarbon oil is 230 parts by mass or less relative to 100 parts by mass of the (b) thermoplastic elastomer.
<7> An anti-delamination agent including:
   (a) a drug;
   (b) a thermoplastic elastomer; and
   (c) non-volatile hydrocarbon oil having a kinematic viscosity of 70 mm²/s or less at 40° C., wherein the (a) drug includes
(i) an amino group that may have a substituent, and
(ii) at least one functional group selected from the group consisting of an ester group, an amide group, an ether group, a ketone group, a thioether group, and an amino group different from the (i) amino group, all of which may have a substituent, where
   the (a) drug has a structure, in which the (i) amino group, and the (ii) at least one functional group are bonded via a C1-C3 hydrocarbon chain, and
   the number of hydroxyl groups per molecule of the (a) drug is 4 or less, and
an amount of the (c) non-volatile hydrocarbon oil is 230 parts by mass or less relative to 100 parts by mass of the (b) thermoplastic elastomer.
<8> A method for suppressing delamination of a patch, including:
using the anti-delamination agent of <7> for a patch.

1. A patch comprising:
an adhesive layer including:
(a) a drug;
(b) a thermoplastic elastomer; and
(c) non-volatile hydrocarbon oil having a kinematic viscosity of 60 mm²/s or less at 40° C., wherein
the (a) drug includes
(i) an amino group that may have a substituent, and
(ii) at least one functional group selected from the group consisting of an ester group, an amide group, an ether group, a thioether group, and an amino group different from the (i) amino group, all of which may have a substituent, where
the (a) drug has a structure, in which the (i) amino group, and the (ii) at least one functional group are bonded via a C1-C3 hydrocarbon chain, and
the number of hydroxyl groups per molecule of the (a) drug is 4 or less, and
an amount of the (c) non-volatile hydrocarbon oil is 230 parts by mass or less relative to 100 parts by mass of the (b) thermoplastic elastomer.

2. The patch according to claim 1,
wherein the (a) drug includes
(i) an amino group that may have a substituent, and
(ii) at least one functional group selected from the group consisting of an ester group, an amide group, and an amino group different from the (i) amino group, all of which may have a substituent,
where the (a) drug has a structure, in which the (i) amino group, and the (ii) at least one functional group are bonded via a C1 or C2 hydrocarbon chain.

3. The patch according to claim 1,
wherein the (b) thermoplastic elastomer includes a styrene-based block copolymer.

4. The patch according to claim 3,
wherein the styrene-based block copolymer includes a mixture of a styrene-isoprene-styrene block copolymer and a styrene-isoprene block copolymer.

5. The patch according to claim 1,
wherein the patch is a matrix patch.

6. A production method of the patch of claim 1, comprising:
mixing
the (a) drug;
the (b) thermoplastic elastomer; and
the (c) non-volatile hydrocarbon oil having a kinematic viscosity of 60 mm²/s or less at 40° C., wherein
the (a) drug includes
(i) an amino group that may have a substituent, and
(ii) at least one functional group selected from the group consisting of an ester group, an amide group, an ether group, a thioether group, and an amino group different from the (i) amino group, all of which may have a substituent, where
the (a) drug has a structure, in which the (i) amino group, and the (ii) at least one functional group are bonded via a C1-C3 hydrocarbon chain, and
the number of hydroxyl groups per molecule of the (a) drug is 4 or less, and
an amount of the (c) non-volatile hydrocarbon oil is 230 parts by mass or less relative to 100 parts by mass of the (b) thermoplastic elastomer.

7. An anti-delamination agent comprising:
(a) a drug;
(b) a thermoplastic elastomer; and
(c) non-volatile hydrocarbon oil having a kinematic viscosity of 60 mm²/s or less at 40° C., wherein
the (a) drug includes
(i) an amino group that may have a substituent, and
(ii) at least one functional group selected from the group consisting of an ester group, an amide group, an ether group, a thioether group, and an amino group different from the (i) amino group, all of which may have a substituent, where
the (a) drug has a structure, in which the (i) amino group, and the (ii) at least one functional group are bonded via a C1-C3 hydrocarbon chain, and
the number of hydroxyl groups per molecule of the (a) drug is 4 or less, and
an amount of the (c) non-volatile hydrocarbon oil is 230 parts by mass or less relative to 100 parts by mass of the (b) thermoplastic elastomer.

8. A method for suppressing delamination of a patch, comprising:
using the anti-delamination agent of claim 7 for a patch.

9. A patch comprising:
an adhesive layer including:
(a) a drug;
(b) a mixture of a styrene-isoprene-styrene block copolymer and a styrene-isoprene block copolymer; and
(c) non-volatile hydrocarbon oil having a kinematic viscosity of 60 mm²/s or less at 40° C., wherein
the (a) drug includes
(i) an amino group that may have a substituent, and
(ii) at least one functional group selected from the group consisting of a hydroxyl group, an ester group, an amide group, an ether group, a ketone group, a thioether group, and an amino group different from the (i) amino group, all of which may have a substituent, where
the (a) drug has a structure, in which the (i) amino group, and the (ii) at least one functional group are bonded via a C1-C5 hydrocarbon chain, and
the number of hydroxyl groups per molecule of the (a) drug is 4 or less, and
an amount of the styrene-isoprene block copolymer in the (b) mixture is 20% by mass or greater.

10. The patch according to claim 9,
wherein the (a) drug includes
(i) an amino group that may have a substituent, and
(ii) at least one functional group selected from the group consisting of a hydroxyl group, an ester group, an amide group, and an amino group different from the (i) amino group, all of which may have a substituent,
where the (a) drug has a structure, in which the (i) amino group, and the (ii) at least one functional group are bonded via a C1 or C2 hydrocarbon chain.

11. The patch according to claim 9,
wherein the patch is a matrix patch.

12. A production method of the patch of claim 9, comprising:
mixing
the (a) drug;
the (b) mixture of the styrene-isoprene-styrene block copolymer and the styrene-isoprene block copolymer; and
the (c) non-volatile hydrocarbon oil having a kinematic viscosity of 60 mm²/s or less at 40° C., wherein
the (a) drug includes
(i) an amino group that may have a substituent, and
(ii) at least one functional group selected from the group consisting of a hydroxyl group, an ester group, an amide group, an ether group, a ketone group, a thioether group, and an amino group different from the (i) amino group, all of which may have a substituent, where
the (a) drug has a structure, in which the (i) amino group, and the (ii) at least one functional group are bonded via a C1-C5 hydrocarbon chain, and
the number of hydroxyl groups per molecule of the (a) drug is 4 or less, and
an amount of the styrene-isoprene block copolymer in the (b) mixture is 20% by mass or greater.

13. (canceled)

14. (canceled)

15. The patch according to claim 1,
wherein the (a) drug is at least one selected from the group consisting of tetracaine, lidocaine, prilocaine, and pharmaceutically acceptable salts of the tetracaine, the lidocaine, and the prilocaine.

16. The production method according to claim 6,
wherein the (a) drug is at least one selected from the group consisting of tetracaine, lidocaine, prilocaine, and pharmaceutically acceptable salts of the tetracaine, the lidocaine, and the prilocaine.

17. The anti-delamination agent according to claim 7,
wherein the (a) drug is at least one selected from the group consisting of tetracaine, lidocaine, prilocaine, and pharmaceutically acceptable salts of the tetracaine, the lidocaine, and the prilocaine.

18. The patch according to claim 9,
wherein the (a) drug is at least one selected from the group consisting of tetracaine, lidocaine, prilocaine, and pharmaceutically acceptable salts of the tetracaine, the lidocaine, and the prilocaine.

19. The production method according to claim 12,
wherein the (a) drug is at least one selected from the group consisting of tetracaine, lidocaine, prilocaine, and pharmaceutically acceptable salts of the tetracaine. the lidocaine, and the prilocaine.
